(19) United States
(12) Patent Application Publication
(10) Pub. No.: US 2006/0173297 A1
(43) Pub. Date: Aug. 3, 2006

(54) Imaging method and apparatus for visualizing coronary heart diseases

(76) Inventor: Stefan Popescu, Erlangen (DE)

Correspondence Address: HARNESS, DICKEY & PIERCE, P.L.C., P.O.BOX 8910, RESTON, VA, 20195, US

(21) Appl. No.: 11/274,205

(22) Filed: Nov. 16, 2005

(30) Foreign Application Priority Data
Nov. 17, 2004 (DE) 10 2004 055 461.7

(51) Int. Cl. A61B 6/00

(52) U.S. Cl. 600/431

(57) ABSTRACT

An imaging method and an apparatus are disclosed for visualizing coronary heart diseases. An imaging tomographic technique is used to record and reconstruct one or more images of the heart or a region of the heart after a contrast agent injection. In the method, in a late period after the contrast agent injection, at least one first image of at least one part of the myocardium is recorded in which a maximum in a contrast agent flow caused by the contrast agent injection occurs, or is to be expected, in the myocardium. Subsequently the first image and/or at least one image derived from the first image for the purpose of better recognizability of a local undersupply of the myocardium are/is displayed. The method and/or the associated apparatus may enable the detection of vascular occlusions or vascular constrictions even in the case of relatively small vessels, which cannot be resolved with the aid of the conventional coronary CTA.

The present application hereby claims priority under 35 U.S.C. §119 on German patent application number DE 10 2004 055 461.7 filed Nov. 17, 2004, the entire contents of which is hereby incorporated herein by reference.

### FIELD

The present invention generally relates to an imaging method for visualizing coronary heart diseases. For example, it may relate to a method in the case of which an imaging tomographic technique, such as the technique of computer tomography, is used to record and reconstruct one or more images of the heart or a region of the heart after a contrast agent injection. The invention also generally relates to an apparatus for carrying out the method.

### BACKGROUND

Imaging techniques for visualizing coronary heart diseases, in particular coronary calcification or strictures, constitute an important aid in evaluating the state of the heart. This relates both to preliminary examinations for the early recognition of circulation disturbances, and to the monitoring of a coronary heart disease, if appropriate after a bypass operation or an angioplasty, over a relatively long period. It is possible with the aid of such examinations to better estimate the risk of a heart attack, and to check the success of an operation or a therapy.

At present, it is preferred to make use for this purpose chiefly of noninvasive imaging techniques such as computed tomography (CT), magnetic resonance tomography (MR) or positron emission tomography (PET). However, measuring cardiac perfusion by use of PET is very cost intensive and delivers only a very low spatial resolution.

Coronary CT angiography (CTA) can be used to record images of the vessel tree of the heart after a contrast agent injection in which vascular constriction can be detected. However, the spatial resolution is still limited in this technique as well. A reliable statement on vascular constrictions can therefore no longer be made from such images for volumes of less than 1 mm³ such as occur in the case of vascular constrictions in peripheral coronary artery segments such as RCA1-4, LM5, LAD6-9 or CX with lumen diameters down to 1 mm.

### SUMMARY

An object of at least one embodiment of the present invention resides in specifying a noninvasive imaging method and/or an apparatus for visualizing coronary heart diseases. From images obtained, it may also be possible to detect vascular constrictions or vascular occlusions that have not so far been capable of detection with the aid of conventional CTA.

An object may be achieved with the aid of the method and/or of the apparatus. Advantageous refinements of the method and of the apparatus can be gathered from the following description and the example embodiments.

In the case of the present imaging method of at least one embodiment, an imaging tomographic technique, in particular the technique of computer tomography, is used to record and reconstruct one or more images of the heart or of a region of the heart after a contrast agent injection. The method is distinguished in that at least one image, designated below as first image, of at least one part of the myocardium is automatically recorded in a late period after the contrast agent injection in which a maximum in a contrast agent flow caused by the contrast agent injection occurs, or is to be expected, in the myocardium. The reconstructed first image and/or at least one image derived from the first image for the purpose of better detectability of a local undersupply or perfusion disturbance of the myocardium are subsequently displayed.

By contrast with the known CTA, in the case of which one or more images of the vessel tree of the heart are recorded in an early period after the contrast agent injection in which the maximum in the contrast agent flow in the vessels of the vessel tree occurs, in the method of at least one embodiment, the recording of the first image is begun at a late instant. This first image therefore does not show the highest contrast in the vessel tree, but in the heart muscle, the myocardium.

In the display of this first image, it is possible, given the occurrence of coronary perfusion disturbances, to detect regions of the myocardium to which no contrast agent, or a substantially lesser fraction of contrast agent, has yet penetrated at this late instant of image recording by contrast with the other regions of the myocardium. Starting from these undersupplied or defectively perfused areas of the myocardium, it is possible to infer vascular occlusions or restrictions of the vessels supplying said areas. Use is made in this case of the fact that partial or complete arterial occlusions influence large regions of the myocardium that lie downstream of the respective occlusion.

In the case of multiple capillary occlusions, the influenced areas lie underneath the capillary network. The direct effect of a vascular constriction, for example owing to calcification, consists in the reduction of the blood flow in the myocardium region affected thereby, which is substantially larger than the region of the vascular occlusion itself. Such undersupplied or poorly perfused areas can therefore already be detected with a substantially lesser spatial resolution of the imaging tomographic technique than would be required for detecting the positive arterial occlusion, for example by use of coronary CTA.

By contrast with a conventional coronary CTA, the recording of the first image requires merely the selection of a longer time interval from the start of the contrast agent injection. This can be performed automatically by presetting in the case of the present method of at least one embodiment. Alternatively, it is also possible after the contrast agent injection to carry out test scans in short time intervals with the aid of which the correct instant for starting the image recording for the first image is determined.

In a preferred refinement of at least one embodiment of the present method, the mode of procedure is firstly as with the conventional coronary CTA, such that at least one image of the vessel tree, designated as second image in at least one embodiment of the present patent application, is recorded with maximum contrast in an early period after the start of the contrast agent injection. Subsequently, after a further delay time the first image is recorded at an instant at which the blood enriched with the contract agent penetrates into the myocardium. Myocardium regions lying downstream of a vascular constriction or of a vascular occlusion are penetrated in this case by contrast agent later, or only to a slight extent. Consequently, with this conduct of the method, the already known technique of coronary CTA is extended by a further step of recording an image of the myocardium with a further time delay in order to track the further propagation of the contrast agent.

Three-dimensional pixel matrices of the heart are preferably generated in each case from the one or more second images and from the one or more first images. In the case of recording 3D volumetric images, these pixel matrices are already present as image data. In the case of recording a number of tomograms, the pixel matrices are compiled from these tomograms and the known spacings of the individual slices. The 3D pixel matrix from the second image and the 3D pixel matrix from the first image are then subtracted from one another. This results in a subtracted pixel matrix in which the regions of the myocardium affected by a vascular occlusion or a vascular constriction can be detected more effectively. The image in this case displays a three-dimensional representation of the perfusion in the myocardium. Such an image can, for example, be recorded repeatedly at different times in the disease process or convalescence process of a patient, in order to be able to compare changes directly.

In a further advantageous development, the subtracted 3D pixel matrix is superposed in the display on a surface image of the heart that can be obtained from the second image by segmentation.

Furthermore, defectively perfused regions and/or effectively perfused regions of the myocardium can be segmented in the first image or in the image derived therefrom, and can be marked in the pictorial display in another color. Thus, the undersupplied regions can be visualized in red, for example, and the normally supplied regions can be visualized in green, for example.

Since the difference between perfused and non-perfused regions of the myocardium is relatively small in the HU values of a CT recording, the image recording and image reconstruction for increasing the accuracy should be carried out in each case on the basis of a 360° scan. In order to reduce the movement artifacts, a computer tomograph having two or more recording systems may be used for example, each including x-ray source and x-ray detector, with the aid of which recording can be performed simultaneously. The image data can be acquired more quickly in this way. An example for such a computer tomograph is to be found, for example, in DE 103 02 565 A1.

In a further preferred refinement, a computer tomograph that enables measurement with the aid of different x-ray spectra is used. Here, different x-ray spectra are used in each case to record at least two first images from which a better separation between perfused and non-perfused regions of the myocardium is possible through the use of the spectral information. The perfused and/or defectively perfused regions area correspondingly segmented and marked in the pictorial display. So as to use the spectral information, it is, for example, possible firstly to carry out the so called rho-Z projection as described, for example, in B. J. Heismann et al., “Density and atomic number measurements with spectral x-ray attenuation method”, Journal of Applied Physics, Volume 94, Number 3, pages 2073-2079. The segmentation can then be performed by windowing the Z-values of the spatial distribution of the effective atomic number Z than are obtained from the rho-Z projection.

In addition to the tomographic imaging system, at least one embodiment of the present apparatus for carrying out the method, for example a computer tomograph, has a control device for image recording that triggers the image recording of at least one first image in a late period after a contrast agent injection in which a maximum in a contrast agent flow caused by the contrast agent injection occurs, or is to be expected, in the myocardium. The apparatus further includes an evaluation device that reconstructs the first image and displays the first image itself or an image derived therefrom. The triggering of the image recording for the first image can be performed by a delay time that is preset or determined online in relation to the start signal of the contrast agent injection.

For the online determination, the control device controls the tomographic imaging system to carry out a number of consecutive test scans from which the matching delay instant for the image recording of the first image is determined by the evaluation device. The evaluation device is respectively designed in this case in the different refinements of the apparatus such that it carries out the image processing in accordance with the previously explained refinements of at least one embodiment of the method.

### BRIEF DESCRIPTION OF THE DRAWINGS

The present method and the associated apparatus are explained again below the aid of an example embodiment in conjunction with the drawings, without limiting the protective scope prescribed by the patent claims. In the drawings:

FIG. 1 shows an example of the schematic design of a computer tomograph for carrying out at least one embodiment of the present method,

FIG. 2 shows an example of the individual method steps in carrying out at least one embodiment of the present method, and

FIG. 3 shows an example of the display of an image obtained in accordance with at least one embodiment of the method.

### DETAILED DESCRIPTION OF THE EXAMPLE EMBODIMENTS

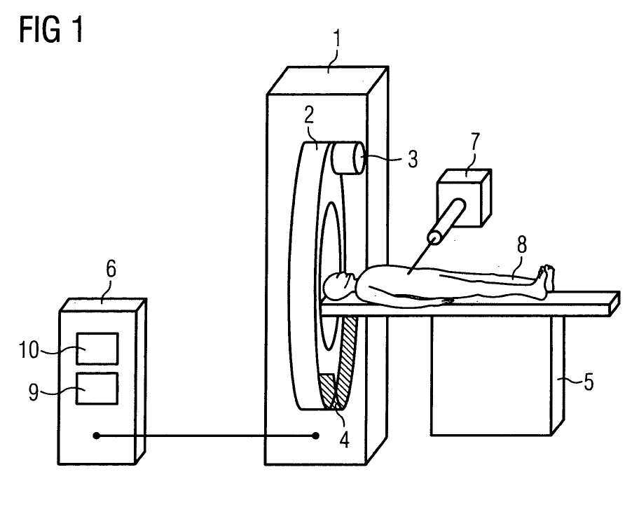

FIG. 1 shows a schematic of a computer tomograph that is designed for carrying out at least one embodiment of the present method. In a known way, the computer tomograph includes, inter alia, an x-ray tube 3, x-ray detectors 4 arranged in the form of rows, and a patient support table 5. The x-ray tube 3 and the x-ray detectors 4 are arranged on the rotating part 2 of a gantry that rotates about the patient support table 5 or an examination axis running parallel thereto. As a rule, the patient support table 5 can be displaced relative to the gantry along the examination axis.

The x-ray tube 3 produces an x-ray beam that is expanded in the form of a fan in a cutting plane perpendicular to the examination axis, and which, during examinations, penetrates a slice of the patient 8, supported on the patient support table 5, and strikes the x-ray detectors 4 located opposite the x-ray tube 3. The angle at which the x-ray beam penetrates the body slice of the patient 8 and, if appropriate, the position of the patient support table 5 relative to the gantry vary continuously during the imaging with the aid of the computer tomograph. During imaging, the x-ray detectors 4 therefore supply a large quantity of measured data that must be evaluated for reconstructing a two-dimensional tomogram or a three-dimensional image of a body region of the patient 8. As a rule, this evaluation is performed in a stationary computer system 6 that is connected to the stationary part 1 of the computer tomograph. The computer system 6 further includes a control device 9 that serves to drive the computer tomograph for carrying out the measurement scan for an image recording, and an evaluation device 10 for evaluating the measured data obtained by the computer tomograph.

Carrying out at least one embodiment of the present method proceeds from the use of a contrast agent that is injected into the elbow vein of the patient 8 with the aid of a contrast agent injector 7. The start of this injection is prompted or detected by the control device 9. Proceeding from this starting instant, the control device 9 controls the computer tomograph after a predetermined delay time to carry out a measurement scan for an image of the patient's heart.

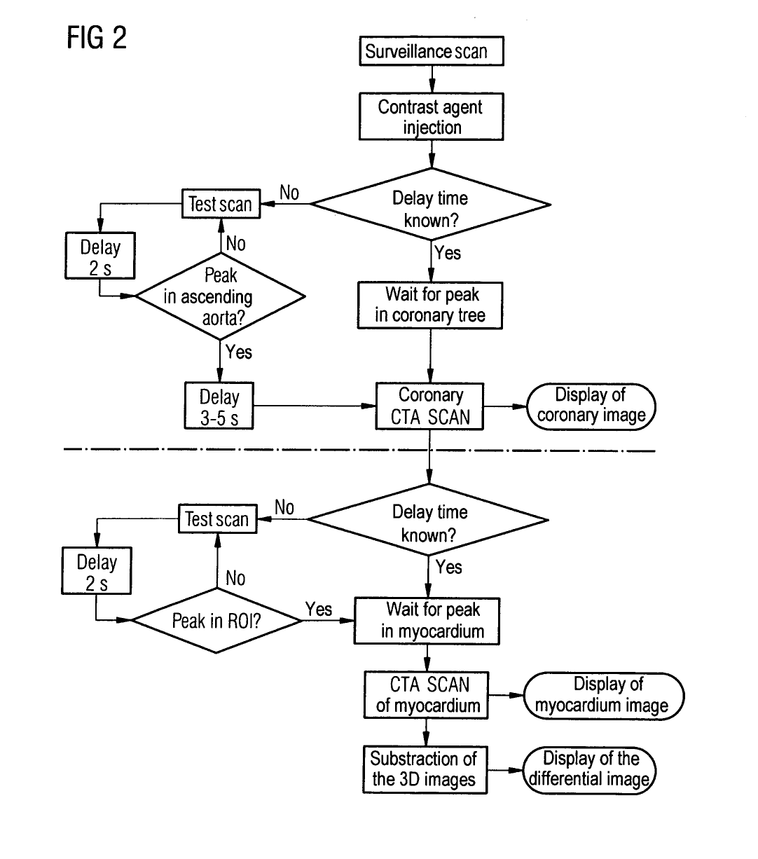

Here, in the present example, the control device is designed such that it prompts a recording of an image of the heart (second image) in an early period after the contrast agent injection, in which a maximum in the contrast agent flow occurs, or is to be expected, in the vessel tree, and a further image of the heart (first image) in a later period after the contrast agent injection during which a maximum in the contrast agent flow is already propagating in the myocardium. This is explained in more detail with the aid of FIG. 2.

FIG. 2 shows here an exemplary method cycle that comprises the carrying out of at least one embodiment of the present method.

Firstly, a surveillance scan of the body region to be examined is carried out by the computer tomograph in order to fix the scan boundaries for the subsequent image recording. The contrast agent is injected after this fixing. Proceeding from this start instant, a coronary CT measurement scan is carried out after a specific time for the purpose of recording the second image of the coronary vessel tree. This mode of procedure corresponds to the known technique of coronary CTA.

The image thereby required, in which the vessels of the vessel tree are visible with maximum contrast, can be displayed on a monitor. The delay time between the start instant of the injection of the contrast agent and the beginning of the measurement scan for recording the second image can either be already known and thus prescribed or, as may be seen in the left-hand part of the figure, be determined during the examination. In the latter case, test scans are carried out after the contrast agent injection, preferably to obtain a tomogram in an axial perspective. At a time interval of, for example, 2 s in each case, it is checked automatically whether a rise in the measurement signal can be recognized in the descending aorta in the tomogram. If this rise is detected, the measurement scan for recording the second image is begun after a further delay of approximately 3-5 s. At this time, the maximum in the contrast agent flow is to be expected in the coronary vessel tree and in the ventricles.

Following a further delay time after the imaging of the second image, the first image for displaying the myocardium is recorded with the aid of a measurement scan. This first image can be likewise be displayed on a monitor. The delay time is selected here such that the first image is recorded at an instant when a maximum in the contrast agent flow occurs in the myocardium. This delay time can either be known in advance and therefore be prescribed, or can be acquired by way of a test scan as in the determination of the first delay time. For this purpose, the measurement signal of the test scan in the myocardium region of interest is monitored in the tomogram at regular time intervals of, for example, 2 s, and the recording of the first image is started in the event of a rise.

The image reconstruction for displaying the images is performed in each case in the evaluation unit of the computer tomograph. It is also possible in this evaluation unit to generate a 3D image, that is to say a 3D pixel matrix, from the second and first image. The two 3D pixel matrices obtained thereby are subtracted from one another pixel by pixel and the differential image is displayed. In this differential image, a 3D image or a 3D pixel matrix show regions of the myocardium that because of their good perfusion have a high fraction of contrast agent, a higher contrast than regions with lower perfusion. Tissue with low perfusion that surrounds the cardiac muscle or regions of the cardiac muscle with low perfusion is eliminated by the subtraction. A three-dimensional image of the perfusion in the myocardium is obtained in this way.

At least one embodiment of the method explained by the example in FIG. 2, has 3 phases. A first phase includes the known coronary CTA; in the second phase, a measurement scan is carried out in accordance with at least one embodiment of the present method in order to record the first image of the myocardium; the 3D subtraction image is calculated in the subsequent third phase.

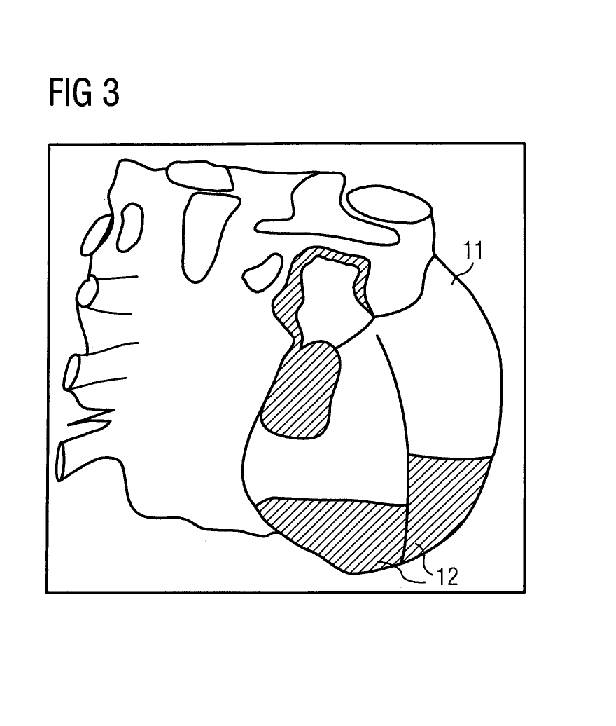

FIG. 3 shows a very schematic example of such an image in which the well perfused regions of the myocardium 11 are indicated by light areas, and the poorly perfused regions 12 are indicated by hatched areas. The display can be done here in a color coded fashion, the hatched zones being able, for example, to have a dark red color, in order to visualize the defective perfusion.

At least one embodiment of the present method can be used for more effective detection of non-calcified coronary lesions without this requiring a higher spatial resolution of the imaging tomographic method, or the use of a higher x-ray dose. At least one embodiment of the present method can easily be converted by extending the known coronary CTA by a further, again delayed image recording step. Because of the lesser demands on the spatial resolution, the sensitivity to movement artifacts is also much reduced.

In the case of recording the first (later) image, the resolution can also be reduced by comparison with the coronary CTA in order to reduce the x-ray dose additionally required. In a combination of the coronary CTA with at least one embodiment of the present method of recording the first image, the action of occlusions recognized in the second image can be checked.

The first image can also be recorded by way of ECG-triggered slice scans with low x-ray dose. It is also possible to use the first image to record just one region of the myocardium in which the effects of vascular occlusions or vascular constrictions are presumed. Above all, it is possible to use at least one embodiment of the method to recognize vascular occlusions or constrictions in relatively small vessels that cannot be resolved with the aid of the conventional coronary CTA.

Example embodiments being thus described, it will be obvious that the same may be varied in many ways. Such variations are not to be regarded as a departure from the spirit and scope of the present invention, and all such modifications as would be obvious to one skilled in the art are intended to be included within the scope of the following claims.

What is claimed is:

1. An imaging method for visualizing coronary heart diseases, wherein an imaging tomographic technique is used to record and reconstruct one or more images of at least one of the heart and a region of the heart after a contrast agent injection, the method comprising:
recording, in a late period after the contrast agent injection, at least one first image of at least one part of the myocardium in which a maximum in a contrast agent flow caused by the contrast agent injection occurs, or is to be expected, in the myocardium; and
displaying at least one of the first image and at least one image derived from the first image for the purpose of better recognizability of a local undersupply of the myocardium.

2. The method as claimed in claim 1, wherein, in addition at least one second image of at least one part of the vessel tree of the heart is recorded in an early period after the contrast agent injection in which a maximum in the contrast agent flow in the vessel tree occurs or is to be expected.

3. The method as claimed in claim 2, wherein the images of identical regions of the heart are recorded, a three-dimensional pixel matrix of the heart is generated in each case from the images, the pixel matrix of the second image and the pixel matrix of the first image are subtracted from one another, and a subtracted pixel matrix obtained therefrom is displayed.

4. The method as claimed in claim 3, wherein a heart surface image is segmented from the second image, and the subtracted pixel matrix is displayed with the heart surface image superposed.

5. The method as claimed in claim 1, wherein regions of the myocardium that are at least one of locally undersupplied and not undersupplied are segmented in at least one of the first image and an image derived therefrom and are color coded in the display of at least one of the first image and the image derived therefrom.

6. The method as claimed in claim 1, wherein the one or more images are recorded with the aid of a computer tomograph that has a number of recording systems composed of x-ray source and x-ray detector for simultaneous image recording, the images being obtained by a 360° image recording and 360° image reconstruction.

7. The method as claimed in claim 1, wherein the at least one first image is recorded multiply and with the aid of different x-ray spectra, and at least one of undersupplied and not undersupplied regions of the myocardium are segmented by using spectral information.

8. An apparatus, comprising:
a tomographic image recording system;
a control device that triggers an image recording at least of one first image in a late period after a contrast agent injection in which a maximum in a contrast agent flow caused by the contrast agent injection occurs, or is to be expected, in the myocardium; and
an evaluation device that reconstructs the first image and displays at least one of the first image and an image derived therefrom.

9. The apparatus as claimed in claim 8, wherein the control device is designed such that it triggers an image recording at least of one second image in an early period after the contrast agent injection in which a maximum in the contrast agent flow occurs, or is to be expected, in the vessel tree of the heart.

10. The apparatus as claimed in claim 9, wherein the evaluation device is designed such that it generates a three-dimensional pixel matrix of the heart from the images in each case, subtracts the pixel matrix of the second image and the pixel matrix of the first image from one another, and displays a subtracted pixel matrix obtained therefrom.

11. The apparatus as claimed in claim 8, wherein the evaluation device is designed in such a way that it segments at least one of locally undersupplied and not undersupplied regions of the myocardium in at least one of the first image and an image derived therefrom, and color codes them for the display of at least one of the first image and the image derived therefrom.

12. The apparatus as claimed in claim 8, further comprising a number of recording systems including an x-ray source and x-ray detector for simultaneous image recording.

13. The apparatus as claimed in claim 8, wherein the apparatus enables image recording with the aid of different x-ray spectra.

14. The apparatus as claimed in claim 9, wherein the evaluation device is designed in such a way that it segments at least one of locally undersupplied and not undersupplied regions of the myocardium in at least one of the first image and an image derived therefrom, and color codes them for the display of at least one of the first image and the image derived therefrom.

15. The apparatus as claimed in claim 10, wherein the evaluation device is designed in such a way that it segments at least one of locally undersupplied and not undersupplied regions of the myocardium in at least one of the first image and an image derived therefrom, and color codes them for the display of at least one of the first image and the image derived therefrom.

16. The apparatus as claimed in claim 8, wherein the apparatus is a computer tomograph.

17. An apparatus, comprising:
means for recording, in a late period after the contrast agent injection, at least one first image of at least one part of the myocardium in which a maximum in a contrast agent flow caused by the contrast agent injection occurs, or is to be expected, in the myocardium; and
means for displaying at least one of the first image and at least one image derived from the first image for the purpose of better recognizability of a local undersupply of the myocardium.

18. The apparatus as claimed in claim 17, wherein the control device is designed such that it triggers an image recording at least of one second image in an early period after the contrast agent injection in which a maximum in the contrast agent flow occurs, or is to be expected, in the vessel tree of the heart.

19. The apparatus as claimed in claim 18, wherein the evaluation device is designed such that it generates a three-dimensional pixel matrix of the heart from the images in each case, subtracts the pixel matrix of the second image and the pixel matrix of the first image from one another, and displays a subtracted pixel matrix obtained therefrom.

20. The apparatus as claimed in claim 17, wherein the evaluation device is designed in such a way that it segments at least one of locally undersupplied and not undersupplied regions of the myocardium in at least one of the first image and an image derived therefrom, and color codes them for the display of at least one of the first image and the image derived therefrom.
